(19) United States
(12) Patent Application Publication
(10) Pub. No.: US 2025/0120761 A1
(43) Pub. Date: Apr. 17, 2025

(54) Handheld Devices and a Method to Achieve Targeted Fat Removal with Minimal Invasiveness

(71) Applicants: Roberto Magana, Cos Cob, CT (US); Rafael Magana, Cos Cob, CT (US)

(72) Inventors: Roberto Magana, Cos Cob, CT (US); Rafael Magana, Cos Cob, CT (US)

(21) Appl. No.: 18/918,011

(22) Filed: Oct. 16, 2024

Related U.S. Application Data
(60) Provisional application No. 63/544,605, filed on Oct. 17, 2023.

(51) Int. Cl. A61B 18/00

(52) U.S. Cl. CPC A61B 18/00; A61B 2018/00464

(57) ABSTRACT

A novel approach that combines technologies to achieve targeted fat removal with minimal invasiveness. The procedure selectively disrupts only fatty tissue, avoiding damage to surrounding tissues, muscles, and minimizing pain and bleeding. Consequently, recovery time is significantly reduced, and the overall procedure becomes safer. In the majority of cases, postoperative narcotic treatment is unnecessary. An additional key feature of this method is the utilization of ultrasound during abdominal suction lipectomy to detect clinically undetectable hernias and for enhancing areas with fat transfer. The method follows a systematic process that involves marking, desensitizing, and anesthetizing the treatment areas using a timed CO2 skin coolant and jet injection delivered through a single handpiece. This method employs specialized devices and a systematic approach to achieve the most minimally invasive, gentle, and pain-free fat removal.

### SEQUENCE LISTING OR PROGRAM

Not Applicable

### TECHNICAL FIELD OF THE INVENTION

The present invention relates to devices and methods to provide fat removal. More particularly, the invention relates to devices and a method to achieve targeted fat removal with minimal invasiveness.

### BACKGROUND OF THE INVENTION

During invasive fat removal procedures, a surgeon makes small incisions in the targeted area. Then, specialized tools such as cannulas are used to break up and suction out unwanted fat cells. The surgeon carefully sculpts the region to achieve the desired contour. Invasive techniques are known for providing more immediate and dramatic results compared to non-invasive alternatives. Examples of these procedures are the tummy tuck or the stomach lift. While these procedures are generally effective, there are several pros and cons to invasive fat removal including associated risks and potential discomfort, longer recovery time compared to non-invasive techniques, higher cost, and potential for scarring.

Non-invasive fat removal refers to procedures that do not require surgical incisions or anesthesia. These treatments use advanced technologies to target and eliminate subcutaneous fat cells without disrupting the surrounding tissues. Non-invasive procedures are generally considered safer and require little to no downtime compared to invasive options.

The main difference between invasive and non-invasive procedures is that the latter offers a more effortless experience, but the downside is that results pale by comparison.

Non-invasive fat removal procedures utilize various technologies such as cold temperatures, laser energy, or ultrasound waves to target and destroy fat cells indirectly. These treatments penetrate the skin without causing damage, allowing the body to eliminate the destroyed fat cells over time naturally.

However, nonsurgical techniques only provide temporary changes, as they cause fat cells to empty their contents instead of physically removing them. Liposuction, by contrast, permanently lowers the concentration of fat cells in target areas, as these do not grow back in adults.

Therefore, what is needed is a method that employs specialized devices and a systematic approach to achieve the most minimally invasive, gentle, and pain-free fat removal.

What is also needed is a procedure that selectively disrupts only fatty tissue, avoiding damage to surrounding tissues, muscles, and minimizing pain and bleeding. Consequently, recovery time is significantly reduced, and the overall procedure becomes safer. In the majority of cases, postoperative narcotic treatment is unnecessary.

### SUMMARY OF THE INVENTION

The devices and methods presented teach a novel approach that combines technologies to achieve targeted fat removal with minimal invasiveness. By eliminating the need for general anesthesia, it significantly reduces associated risks. The procedure selectively disrupts only fatty tissue, avoiding damage to surrounding tissues, muscles, and minimizing pain and bleeding. Consequently, recovery time is significantly reduced, and the overall procedure becomes safer. In the majority of cases, postoperative narcotic treatment is unnecessary.

An additional key feature of this method is the utilization of ultrasound during abdominal suction lipectomy to detect clinically undetectable hernias and for enhancing areas with fat transfer.

The method follows a systematic process that involves marking, desensitizing, and anesthetizing the treatment areas using a timed CO2 skin coolant and jet injection delivered through a single handpiece.

This method employs specialized devices and a systematic approach to achieve the most minimally invasive, gentle, and pain-free fat removal.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a side planar view of the handheld topical anesthetic jet injector and CO2 cryoanesthesia device taught by the present invention.

FIG. 2 is a front planar view of the handheld topical anesthetic jet injector and CO2 cryoanesthesia device taught by the present invention.

FIG. 3 is a side cross section view of the handheld topical anesthetic jet injector and CO2 cryoanesthesia device taught by the present invention.

FIG. 4 is a side cross section view of a pneumatic version of the handheld topical anesthetic jet injector and CO2 cryoanesthesia device taught by the present invention.

FIG. 5 is a side cross section view of the handheld topical anesthetic jet injector and CO2 cryoanesthesia device taught by the present invention illustrating a removable tip.

FIG. 6 is a flow chart illustrating the method to achieve targeted fat removal with minimal invasiveness taught by the present invention.

FIG. 7 is a biopsy automatic pen as taught by the present invention.

FIG. 8 is a biopsy automatic pen in a cocked potion as taught by the present invention.

FIG. 9 is a biopsy automatic pen with a punch loaded and the device activated having a 7 mm punch tip exposed as taught by the present invention.

FIG. 10 is a pneumatic version of a biopsy automatic pen with a hose to an external tank for power as taught by the present invention.

FIG. 11 is a pneumatic version of a biopsy automatic pen with a hose to an external tank for power in a cocked position as taught by the present invention.

FIG. 12 is a pneumatic version of a biopsy automatic pen with a hose to an external tank for power with a punch loaded and the device activated having a 7 mm punch tip exposed as taught by the present invention.

FIG. 13 is a planar view of the 16 Gauge infiltration cannulas of 20 cm and 25 cm length taught by the present invention.

FIG. 14 is a planar view of the garden spiral cannula with rounded tip taught by the present invention.

FIG. 15 is a planar view of a screw base cannula for creating a natural motion as taught by the present invention.

FIG. 16 is a perspective view of the lymphatic dispersion massage gun taught by the present invention.

FIG. 17 is a back planar view of the lymphatic dispersion massage gun taught by the present invention.

FIG. 18 is a front planar view of the lymphatic dispersion massage gun taught by the present invention.

FIG. 19 is a side planar view of the lymphatic dispersion massage gun taught by the present invention.

FIG. 20 is a top planar view of the lymphatic dispersion massage gun taught by the present invention.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized, and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

The device of the present invention is a handheld topical anesthetic jet injector and CO2 cryoanesthesia device. The handheld topical anesthetic jet injector and CO2 cryoanesthesia device is a cutting-edge medical tool designed to provide efficient and effective topical anesthesia in a convenient and user-friendly manner.

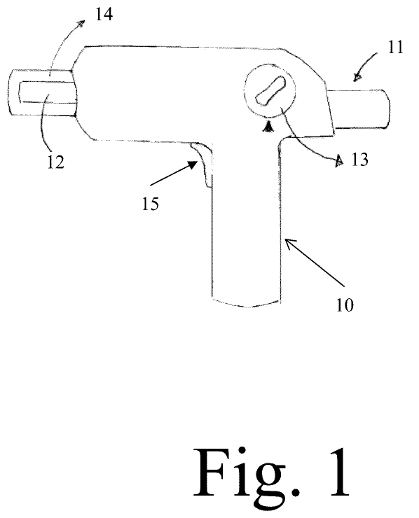

As shown in FIGS. 1-5, the device is shaped like a gun 10. The handheld topical anesthetic jet injector and CO2 cryoanesthesia device combines the benefits of a jet injector and CO2 cryoanesthesia in a single handheld unit, revolutionizing the administration of local anesthesia.

This device seamlessly integrates a jet injector and CO2 cryoanesthesia capabilities into a single, compact unit. It eliminates the need for multiple tools, streamlining the anesthesia process.

FIG. 1 is a side planar view of the handheld topical anesthetic jet injector and CO2 cryoanesthesia device taught by the present invention. The device is shaped like a gun 10 where the front is comprised of a CO2 Gas Dispenser 14 and tip 12. The middle of the gun shaped body houses the timer 13. The handle portion of the gun shaped body provides a handle that makes a trigger 15 accessible when holding the device. The rear portion of the gun shaped body houses the CO2 tank 11.

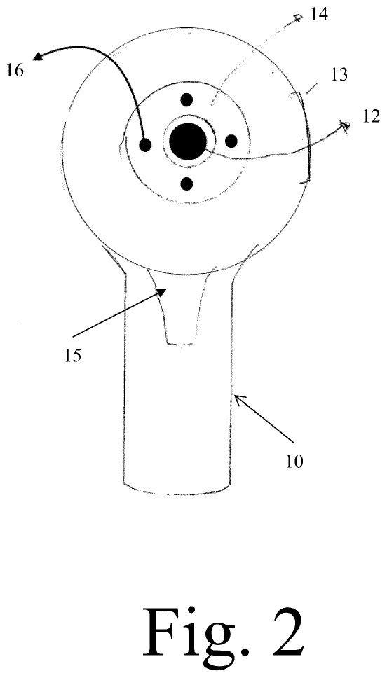

FIG. 2 is a front planar view of the handheld topical anesthetic jet injector and CO2 cryoanesthesia device taught by the present invention. The front planar view of FIG. 2 details the CO2 Gas Dispenser 14 and tip 12 which is further defined by a plurality of CO2 gas outlets 16.

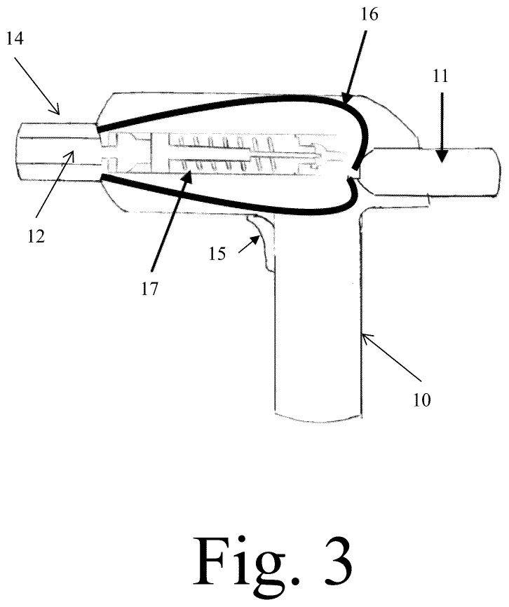

FIG. 3 is a side cross section view of the handheld topical anesthetic jet injector and CO2 cryoanesthesia device taught by the present invention. This cross-section view shows the internal components and their relationship to each other. Between the tip 12 and CO2 Tank 11, within the gun 10 body, one or more CO2 transporting pipes 17 provide a path to transfer CO2 from the tank 11 to the nozzle 12. An injector spring mechanism 18 is also retained within the gun 10 body for delivering the injection when the trigger 15 is squeezed.

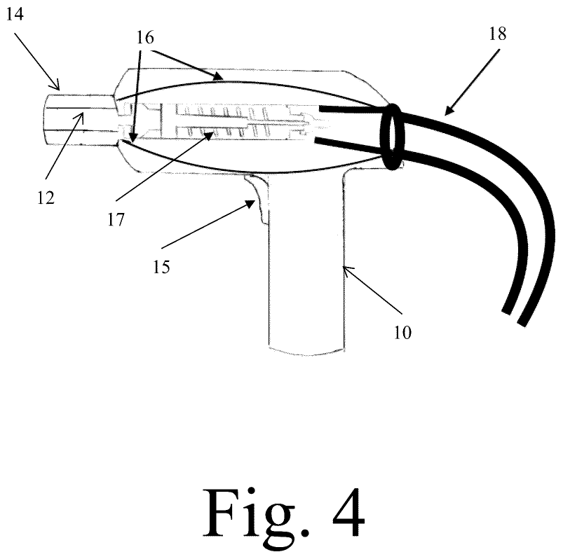

FIG. 4 is a side cross section view of a pneumatic version of the handheld topical anesthetic jet injector and CO2 cryoanesthesia device taught by the present invention. Between the tip 12 and CO2 Tank 11, within the gun 10 body, one or more CO2 transporting pipes 17 provide a path to transfer CO2 from the tank 11 to the nozzle 12. An injector spring mechanism 18 is also retained within the gun 10 body for delivering the injection when the trigger 15 is squeezed. In the embodiment illustrated in FIG. 4, a hose connection to an external CO2 tank for pressure and cooling 18 replaces the tank 11.

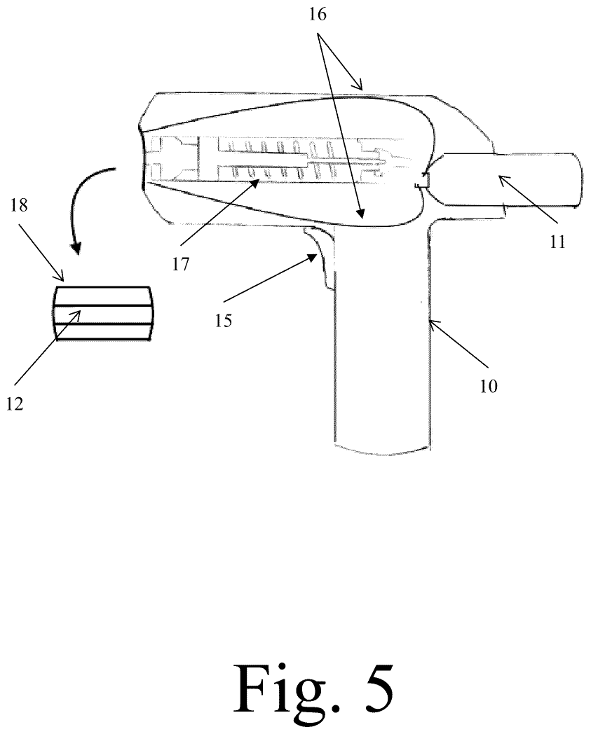

FIG. 5 is a side cross section view of the handheld topical anesthetic jet injector and CO2 cryoanesthesia device taught by the present invention illustrating a removable tip 18. This cross-section view shows the internal components and their relationship to each other. Between the removeable tip 18 and CO2 Tank 11, within the gun 10 body, one or more CO2 transporting pipes 17 provide a path to transfer CO2 from the tank 11 to the nozzle 12. An injector spring mechanism 18 is also retained within the gun 10 body for delivering the injection when the trigger 15 is squeezed.

The tip 12 of the device can be easily removed as illustrated in FIG. 5 as a removable tip 18 for sterilization in an autoclave, ensuring optimal hygiene and preventing cross-contamination between patients.

As shown in FIGS. 1, 3, and 5 the rear section of the device is loaded with a disposable 33g CO2 tank 11, which provides a controlled release of CO2. The front section is loaded with lidocaine, a commonly used topical anesthetic, ready for deployment.

The device is equipped with an adjustable timer 13 shown in FIGS. 1 and 2, allowing healthcare professionals to set the desired duration of CO2 exposure on the skin before initiating lidocaine administration. This feature enables precise control over the anesthesia process.

Upon activation, the CO2 is released through the front nozzle 12 of the device, creating a cooling effect on the skin. This cryoanesthetic exposure numbs the targeted area and prepares it for subsequent lidocaine application.

Once the preprogrammed time for CO2 exposure has lapsed, the jet injector is activated. It deploys lidocaine onto the center of the previously cooled area, ensuring targeted and localized anesthesia.

The combined CO2 cryoanesthesia and jet injection technologies provide a more comfortable and less painful anesthesia experience for patients compared to traditional methods.

The device's streamlined operation reduces the overall time required for topical anesthesia administration, enhancing efficiency in medical procedures.

The adjustable timer 13 allows healthcare professionals to tailor the duration of CO2 exposure and precisely control the depth and area of anesthesia, ensuring optimal results.

The handheld gun-shaped design makes the device easy to handle and maneuver, facilitating quick and accurate administration of topical anesthesia across various medical settings.

The method to achieve targeted fat removal with minimal invasiveness taught by the presents a novel approach that combines technologies to achieve targeted fat removal with minimal invasiveness. By eliminating the need for general anesthesia, the device and method to achieve targeted fat removal with minimal invasiveness taught by the present invention significantly reduces associated risks. The procedure selectively disrupts only fatty tissue, avoiding damage to surrounding tissues, muscles, and minimizing pain and bleeding. Consequently, recovery time is significantly reduced, and the overall procedure becomes safer. In the majority of cases, postoperative narcotic treatment is unnecessary.

An additional key feature of this method is the utilization of ultrasound during abdominal suction lipectomy to detect clinically undetectable hernias and for enhancing areas with fat transfer.

The method to achieve targeted fat removal with minimal invasiveness follows a systematic process that involves marking, desensitizing, and anesthetizing the treatment areas using a timed CO2 skin coolant and jet injection delivered through a single handpiece, the device taught herein.

This method employs specialized devices and a systematic approach to achieve the most minimally invasive, gentle, and pain-free fat removal.

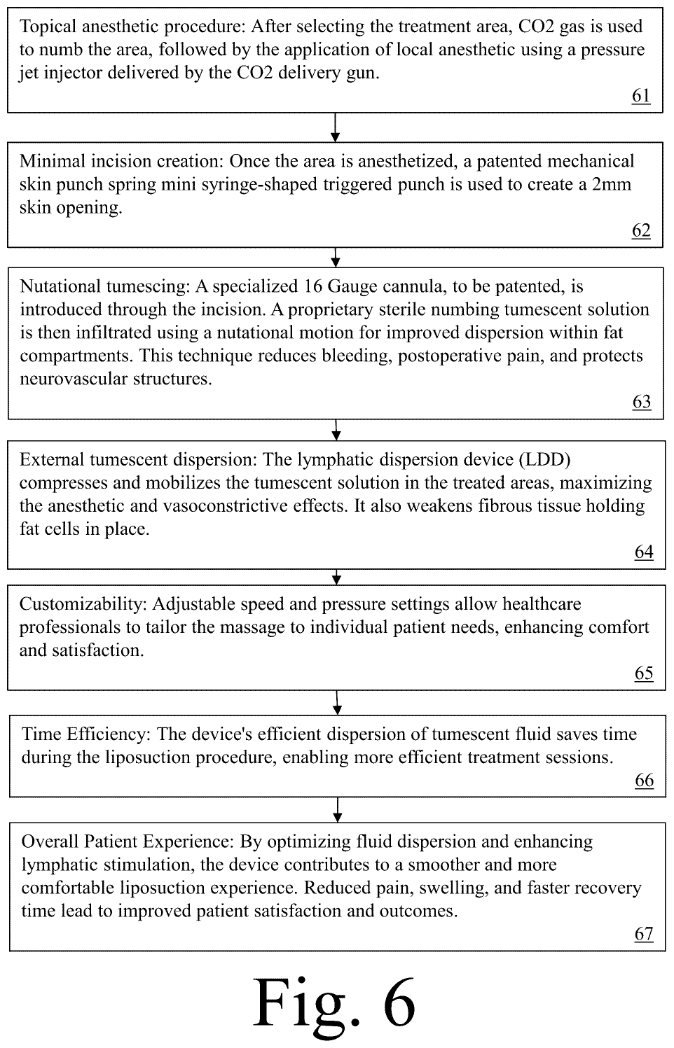

Now referring to FIG. 6, the following sequence of events illustrates the concept and method to achieve targeted fat removal with minimal invasiveness taught by the present invention.

The first step is the topical anesthetic procedure 61 where after selecting the treatment area, CO2 gas is used to numb the area, followed by the application of local anesthetic using a pressure jet injector delivered by the CO2 delivery gun 10 with a CO2 Cartridge cooling mechanism as shown in FIGS. 1-3 or with an external tank 11 cooling system, and pneumatic power as shown in FIGS. 4-5.

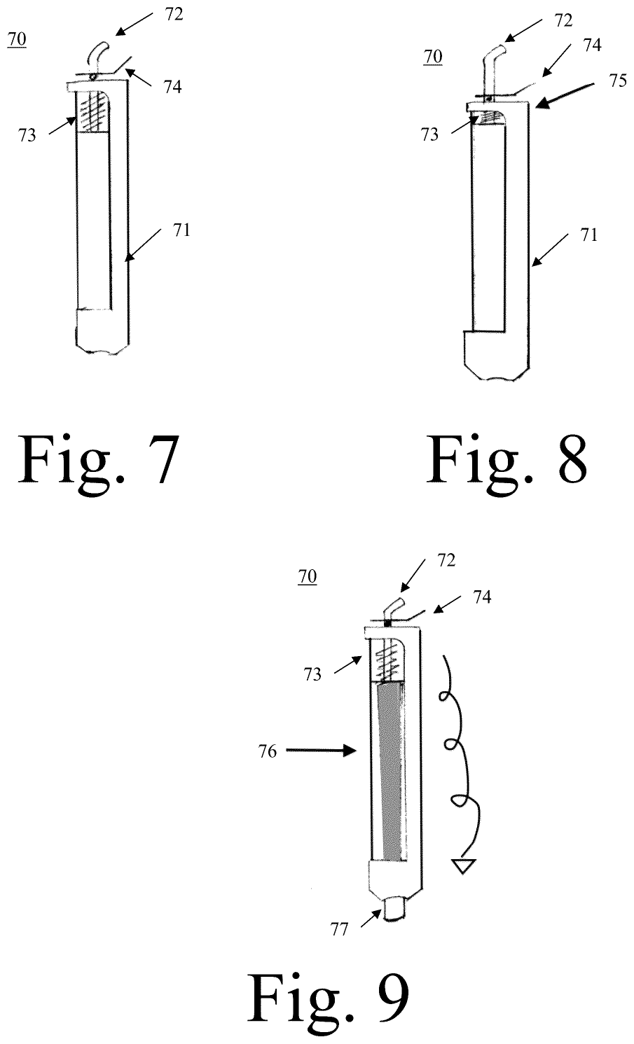

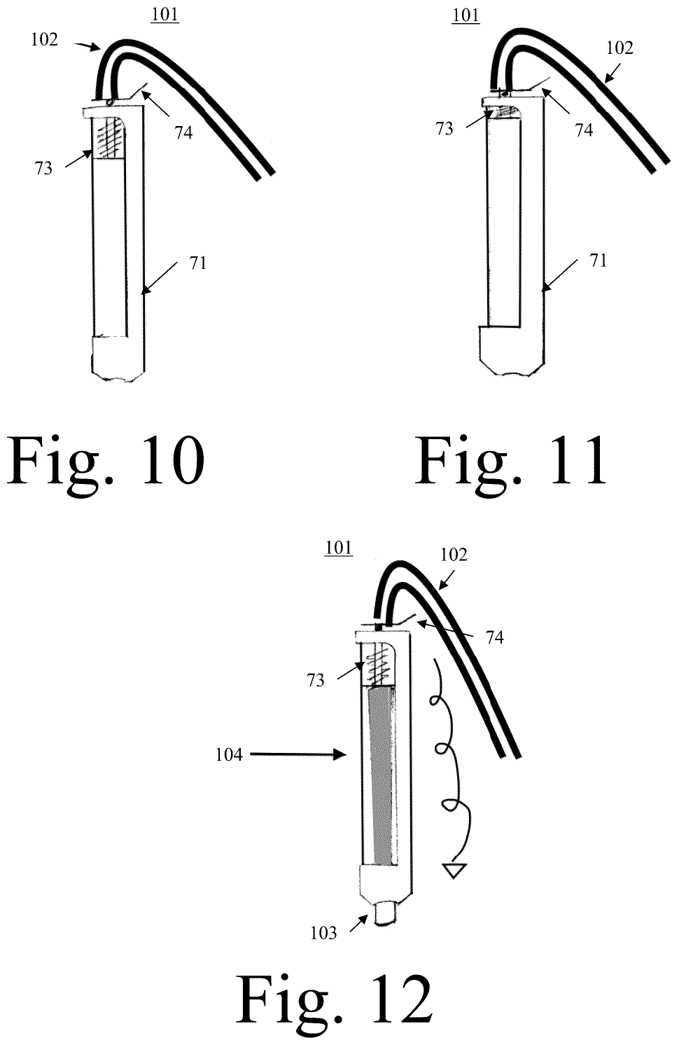

The next step is minimal incision creation 62, wherein once the area is anesthetized, a mechanical skin punch spring mini syringe-shaped triggered punch is used to create a 2 mm skin opening using a spring activated device as shown in FIGS. 7-9 or a pneumatic activated device as shown in FIGS. 10-12.

FIG. 7 is a biopsy automatic pen 70 as taught by the present invention having a body 71 retaining a plunger 72, spring 73, and lock 74.

FIG. 8 is a biopsy automatic pen 70 in a cocked potion 75 as taught by the present invention where the plunger 72 is pulled and compresses the spring 73 and is retained in a locked position by the lock 74 retaining the plunger 72 in a fixed, locked position.

FIG. 9 is a biopsy automatic pen 70 with a punch 76 loaded and the device activated having a 7 mm punch tip 77 exposed as taught by the present invention.

FIG. 10 is a pneumatic version of a biopsy automatic pen 101 with a hose 102 to an external tank for power as taught by the present invention having a body 71 retaining a plunger 72, spring 73, and lock 74.

FIG. 11 is a pneumatic version of a biopsy automatic pen 101 with a hose 102 to an external tank for power in a cocked position as taught by the present invention where the plunger 72 is pulled and compresses the spring 73 and is retained in a locked position by the lock 74 retaining the plunger 72 in a fixed, locked position.

FIG. 12 is a pneumatic version of a biopsy automatic pen 101 with a hose 102 to an external tank for power with a punch loaded and the device activated 104 having a 7 mm punch tip exposed 103 as taught by the present invention.

The handheld biopsy punch pen is a compact and innovative medical device designed for precise and efficient skin biopsy procedures. Shaped like a pen, this handheld tool offers convenience and ease of use, making it an essential instrument for healthcare professionals in dermatology and other related fields.

The device features a cartridge that securely holds a 2 mm biopsy punch. This cartridge system ensures quick and easy replacement of the punch, minimizing downtime during procedures.

The biopsy punch pen incorporates an internal spring and thread mechanism to facilitate controlled advancement and rotation of the punch. This design allows for precise positioning and penetration during the biopsy process.

By cocking the pen, the internal spring recedes, creating space for the biopsy punch to be loaded into position. This mechanism ensures proper alignment and readiness for activation.

When the device is activated, the internal spring propels the biopsy punch forward in a controlled manner. The spring's force enables efficient penetration and sampling of the target tissue.

As the punch moves forward, the internal thread mechanism causes it to spin. This rotational movement helps to minimize tissue trauma and facilitate smooth cutting action during the biopsy procedure.

Once the device is fully activated, the biopsy punch is exposed precisely at a depth of 7 mm. This standardized depth ensures consistent and accurate sampling during each biopsy, enhancing procedural reliability.

After completing the biopsy, the punch can be easily removed from the device for disposal. This eliminates the need for manual extraction and reduces the risk of contamination or accidental injury.

The handheld biopsy punch pen creates a 2 mm hole in the patient's skin, allowing for the extraction of a tissue sample of optimal size for pathological examination.

The combination of spring-driven forward movement and rotational motion ensures precise and consistent biopsy sampling, minimizing the need for re-biopsy and enhancing diagnostic accuracy.

The straightforward cartridge-based design and easy-to-use cocking mechanism streamline the biopsy process, reducing procedure time and improving workflow efficiency.

The use of a 2 mm biopsy punch and controlled penetration depth contributes to minimal trauma and discomfort for the patient, promoting a positive biopsy experience.

The pen-shaped design offers ergonomic handling, making it comfortable to hold and maneuver during procedures. The device's intuitive operation allows healthcare professionals to focus on the biopsy task at hand.

The disposable biopsy punch eliminates the risk of cross contamination and ensures proper infection control practices.

The handheld biopsy punch pen is a reliable and user-friendly tool for dermatologists and healthcare professionals involved in skin biopsy procedures. With its advanced features, precise biopsy sampling, and convenient design, this device enhances the accuracy, efficiency, and patient comfort associated with skin biopsies.

Additionally, the handheld biopsy punch pen is designed to be compatible with autoclave sterilization processes. The materials used in its construction are selected to withstand the high temperatures and pressure of autoclaving, ensuring effective sterilization and maintaining optimal hygiene standards. This feature provides healthcare professionals with the confidence and convenience of using a thoroughly sterilized device for each biopsy procedure, minimizing the risk of contamination and promoting patient safety.

The handheld biopsy punch pen as shown in FIGS. 7-12, will load a disposable punch. When activated, a spring/thread mechanism will move the punch forward, exposing the 7 mm sharp tip. This will slice the tissue instead of cutting with perpendicular pressure. As a result, the patient will feel no pressure associated with the incision.

The third step is a nutational tumescing 63 where a specialized 16 Gauge cannula is introduced through the incision. A proprietary sterile numbing tumescent solution is then infiltrated using a nutational motion for improved dispersion within fat compartments. This technique reduces bleeding, postoperative pain, and protects neurovascular structures.

This cannula already exists, but it does not exist with the screw adapter as shown in FIG. 15.

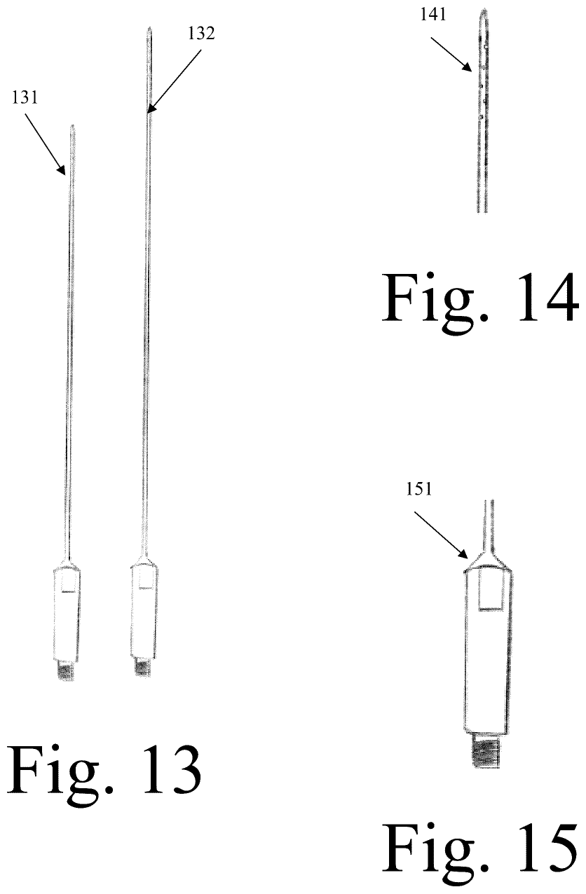

FIG. 13 is a planar view of the 16 Gauge infiltration cannulas of 20 cm 131 and 25 cm length 132 taught by the present invention. FIG. 14 is a planar view of the garden spiral cannula 141 with rounded tip taught by the present invention.

FIG. 15 is a planar view of a screw base cannula 151 for creating a natural motion as taught by the present invention.

The 16 Gauge infiltration cannula 131 for dispersion of tumescent solution into subdermal fat deposits for subsequent fat extraction. Overall, the infiltration cannula designed for nutational motion offers a targeted and efficient method for dispersing tumescent anesthetic fluid during surgical procedures. Its unique mechanism allows for even distribution, enhancing the effectiveness of anesthesia and vasoconstriction while minimizing patient discomfort.

The fourth step is an external tumescent 64 dispersion where the lymphatic dispersion device (LDD) compresses and mobilizes the tumescent solution in the treated areas, maximizing the anesthetic and vasoconstrictive effects. It also weakens fibrous tissue holding fat cells in place.

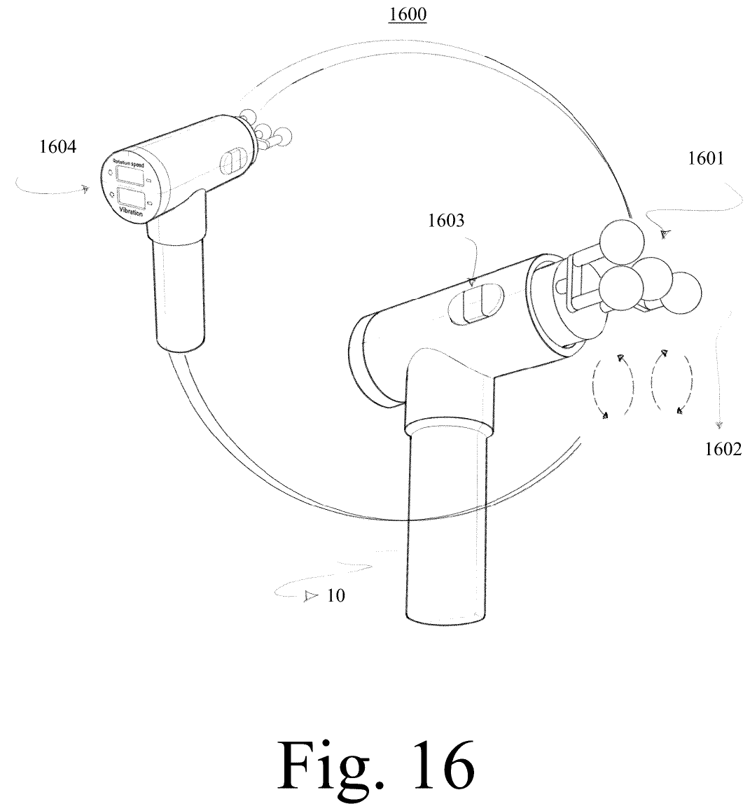

A lymphatic dispersion massage gun 10 taught by the present invention and shown in FIGS. 16-20 is a groundbreaking handheld device that revolutionizes the liposuction process by optimizing the dispersion of tumescent fluid and anesthetic while prioritizing patient safety and comfort. This innovative device features a dual-pair head mechanism that rotates in opposite directions, employing dual-motion technology for superior results.

The device's primary purpose is to efficiently disperse tumescent fluid under the skin and within the fat layer, preparing the area for suctioning during liposuction. The clockwise and counterclockwise rotation of the pairs of heads creates a targeted massaging action that pushes lymphatic material outward, promoting its movement through the patient's own lymphatic drainage system.

The device offers a wide range of uses and benefits for liposuction patients. The dual-pair head mechanism ensures optimal distribution of tumescent fluid, resulting in even and thorough coverage. This promotes effective numbing and swelling reduction during the procedure. The massaging action stimulates the lymphatic system, facilitating the movement of lymphatic fluid. This aids in waste removal, reduces swelling, and improves recovery time. The device promotes the efficient dispersal of anesthetic fluid, ensuring its thorough distribution within the targeted areas. This enhances patient comfort and minimizes pain during the liposuction procedure. Controlled massaging action minimizes the risk of fluid accumulation or uneven distribution, reducing the potential for complications or post-operative issues.

Adjustable speed and pressure settings 65 allow healthcare professionals to tailor the massage to individual patient needs, enhancing comfort and satisfaction.

The device's time efficient dispersion 66 of tumescent fluid saves time during the liposuction procedure, enabling more efficient treatment sessions.

By optimizing fluid dispersion and enhancing lymphatic stimulation 67, the device contributes to a smoother and more comfortable liposuction experience. Reduced pain, swelling, and faster recovery time lead to improved patient satisfaction and outcomes.

Furthermore, the device incorporates a pressure sensor to prevent excessive force on the treatment area, ensuring patient safety. It also includes a vibrating function that enhances effectiveness by increasing blood flow and promoting lymphatic drainage, providing a soothing and relaxing experience during the procedure.

In summary, this innovative handheld device shown in FIGS. 16-20 with its dual-pair head mechanism, pressure sensor, and vibrating function represents a significant advancement in liposuction technology. It optimizes tumescent fluid dispersion, promotes lymphatic stimulation, and prioritizes patient safety and comfort. With its customizable settings and advanced features, it sets a new standard in liposuction treatment, offering superior results and exceptional patient experience.

The device as shown in FIGS. 16-20 has heads or massage tips 1601 that move in opposite directions 1602. By applying the device message tips 1601 to the tumesced area, and moving it in a given direction, the outwards motion 1602 of the device's message tips 1601 will help disperse the tumescent more efficiently through the lymphatic passage ways, resulting in a wider, and deeper dispersion of the anesthetic fluid through the areas to be treated.

FIG. 16 is a perspective view of the lymphatic dispersion massage gun 1600 taught by the present invention. The massage gun 1600 has a plurality of heads or massage tips 1601 that move in opposite directions 1602. A side display 1603 has yellow/green/red lights to display a reading in relation to the amount of pressure being applied by the user to a recipient or patient by the plurality of heads or massage tips 1601 that move in opposite directions 1602. A back display 1604 shows rotation speed and vibration level/setting.

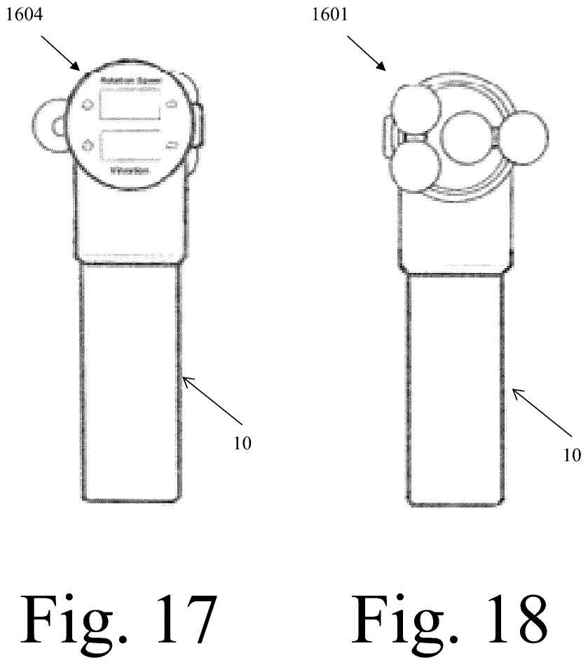

FIG. 17 is a back planar view of the lymphatic dispersion massage gun taught by the present invention. A back display 1604n shows rotation speed and vibration level/setting.

FIG. 18 is a front planar view of the lymphatic dispersion massage gun taught by the present invention. The massage gun 1600 has a plurality of heads or massage tips 1601 that move in opposite directions 1602.

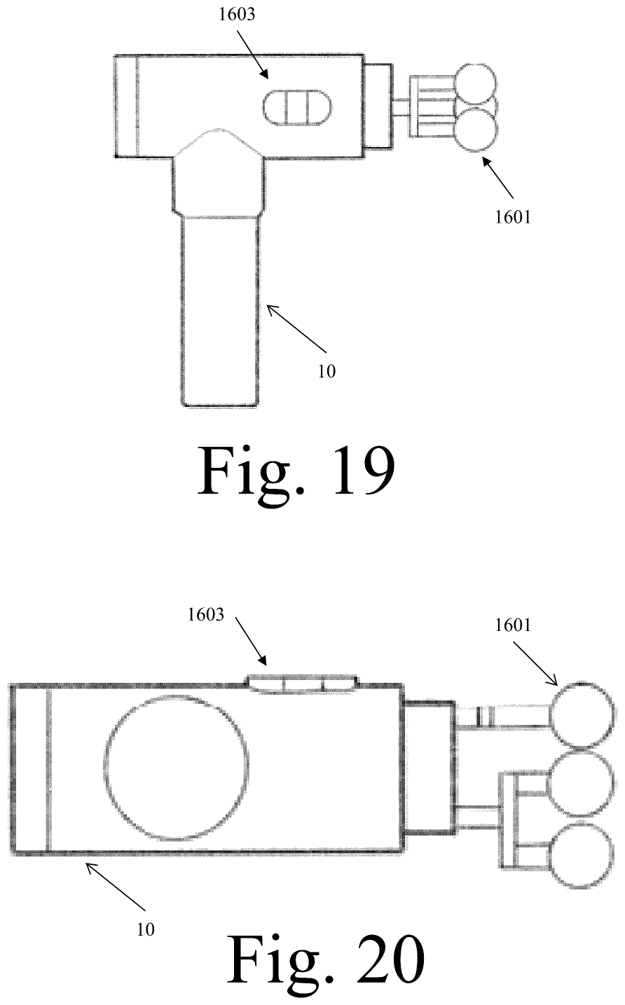

FIG. 19 is a side planar view of the lymphatic dispersion massage gun taught by the present invention. A side display 1603 has yellow/green/red lights to display a reading in relation to the amount of pressure being applied by the user to a recipient or patient by the plurality of heads or massage tips 1601 that move in opposite directions 1602.

FIG. 20 is a top planar view of the lymphatic dispersion massage gun taught by the present invention. A side display 1603 has yellow/green/red lights to display a reading in relation to the amount of pressure being applied by the user to a recipient or patient by the plurality of heads or massage tips 1601 that move in opposite directions 1602.

The fifth step is fat liquefaction and removal where a VASER three-ringed probe is used through the 2 mm port sites created in step 2, for further targeting, emulsification, and liquefaction of fat. VASER liposuction employs ultrasound technology to break apart fat cells and facilitate their removal. The heat generated by the VASER probe also promotes skin tightening by proximity to the reticular dermis

The final step in the process is the progressive fat removal where the removal of fat is carried out in a progressive manner using cannulas specifically designed to gently and selectively extract fat cells. This approach allows for precise fat removal while preserving the integrity of surrounding tissues.

By following this method, targeted fat removal can be achieved with minimal invasiveness, reduced pain, and faster recovery. The combination of technologies, such as CO2 skin coolant, jet injection, nutational tumescing, external tumescent dispersion, and VASER liposuction, ensures a comprehensive and effective fat removal process.

Overall, this novel method offers a safer and more efficient alternative to traditional liposuction procedures. It minimizes the need for general anesthesia, reduces the risk of complications, and delivers satisfactory results in terms of fat reduction and body sculpting.

Thus, it is appreciated that the optimum dimensional relationships for the parts of the invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

1. An apparatus for targeted fat removal, comprising:
a handheld topical anesthetic jet injector and CO2 cryoanesthesia device shaped like a gun;
a removable sterilizable tip for autoclave cleaning;
a rear section containing a disposable 33g CO2 tank; and
a front section loaded with lidocaine for local anesthesia delivery.

2. The apparatus of claim 1, further comprising an adjustable timer for setting the duration of CO2 exposure on the skin prior to administering lidocaine.

3. The apparatus of claim 2, wherein:
upon activation, CO2 is released through the front nozzle, creating a cooling effect on the skin to numb the area; and
a jet injector is activated after the CO2 exposure, deploying lidocaine onto the cooled area for targeted anesthesia.

4. The apparatus of claim 2, wherein the handheld topical anesthetic jet injector and CO2 cryoanesthesia device shaped like a gun consisting of
a handheld biopsy punch pen, comprising:
a cartridge securely holding a 2 mm biopsy punch;
an internal spring and thread mechanism for controlled advancement and rotation of the punch; and
a cocking mechanism to load the punch into position for controlled tissue penetration.

5. The apparatus of claim 4, wherein the biopsy punch pen incorporates an internal spring that recedes when cocked and propels the biopsy punch forward upon activation, enabling efficient penetration of target tissue.

6. The handheld biopsy punch pen of claim 5, further comprising a mechanism that rotates the punch as it moves forward, allowing for precise tissue sampling at a depth of 7 mm.

7. The apparatus for targeted fat removal of claim 1, further comprising
a lymphatic dispersion device (LDD) for fat removal procedures, comprising:
a dual-pair head mechanism that rotates in opposite directions for targeted massaging action;
an adjustable speed and pressure settings for tailoring the massaging action to patient needs; and
a vibrating function to increase blood flow and promote lymphatic drainage.

8. The lymphatic dispersion device (LDD) of claim 7, wherein the dual-pair head mechanism ensures optimal distribution of tumescent fluid through clockwise and counterclockwise rotation, enhancing lymphatic drainage.

9. The lymphatic dispersion device (LDD) of claim 8, further comprising a pressure sensor to prevent excessive force on the treatment area during the procedure, enhancing patient safety.

10. The apparatus for targeted fat removal of claim 1, further comprising
a cannula apparatus, comprising:
a specialized 16 Gauge cannula for use in fat removal procedures; and
a screw adapter for secure attachment and nutational motion to improve tumescent fluid dispersion within fat compartments.

11. A method for targeted fat removal with minimal invasiveness, comprising the steps of:
selecting the treatment area;
numbing the area using CO2 gas delivered by a handheld topical anesthetic jet injector and CO2 cryoanesthesia device;
administering local anesthetic using a pressure jet injector through the CO2 delivery gun;
creating a 2 mm skin opening with a mechanical skin punch;
introducing a 16 Gauge cannula for tumescent infiltration;
compressing and mobilizing the tumescent solution using a lymphatic dispersion device (LDD); and
adjusting the LDD settings to tailor the procedure to patient needs.

12. The method of claim 11, wherein CO2 gas is used to numb the area followed by the application of local anesthetic using a CO2 cartridge cooling mechanism or an external tank cooling system with pneumatic power.

13. The method of claim 11, further comprising the step of using ultrasound during abdominal suction lipectomy to detect clinically undetectable hernias and enhance areas for fat transfer.

14. The method of claim 11, wherein a mechanical skin punch with a spring-activated or pneumatic system is used to create a 2 mm skin opening after the area has been anesthetized.

15. The method of claim 11, further comprising the step of introducing a proprietary sterile numbing tumescent solution using a nutational motion for improved dispersion within fat compartments.

16. The method of claim 11, further comprising the step of fat liquefaction and removal using a VASER three-ringed probe through the 2 mm port sites for emulsification and liquefaction of fat.

17. The method of claim 11, wherein the progressive removal of fat is carried out in a controlled manner using cannulas designed to selectively extract fat cells.

18. The method of claim 11, further comprising the step of using a lymphatic dispersion device (LDD) with dual-pair rotating heads to compress and mobilize the tumescent solution in the treated areas, enhancing lymphatic drainage.

19. The method of claim 11, further comprising the step of using an LDD equipped with a pressure sensor to prevent excessive force during treatment and a vibrating function to enhance lymphatic drainage and blood flow.

20. The method of claim 11, wherein the anesthetic application process is controlled by an adjustable timer on the CO2 cryoanesthesia device, ensuring precise timing of CO2 exposure before administering lidocaine.
